(19) United States
(12) Patent Application Publication
(10) Pub. No.: US 2022/0023425 A1
(43) Pub. Date: Jan. 27, 2022

(54) STRETCHABLE BACKING LAYERS FOR TRANSDERMAL DRUG DELIVERY SYSTEMS

(71) Applicant: Noven Pharmaceuticals, Inc., Miami, FL (US)

(72) Inventors: Keita MORI, Tosu (JP); Takito SHIMA, Ryugasaki (JP); Jun LIAO, Miami, FL (US)

(73) Assignee: Noven Pharmaceuticals, Inc., Miami, FL (US)

(21) Appl. No.: 17/193,223

(22) Filed: Mar. 5, 2021

Related U.S. Application Data
(63) Continuation of application No. 15/351,616, filed on Nov. 15, 2016, now abandoned.
(60) Provisional application No. 62/255,700, filed on Nov. 16, 2015.

(51) Int. Cl. A61K 47/32; A61K 47/44; A61K 9/70; A61K 47/34; A61F 13/02; A61K 31/192; A61M 37/00

(52) U.S. Cl. CPC A61K 47/32; A61K 47/44; A61K 9/703; A61K 47/34; A61K 9/7069; A61K 9/7061; A61K 9/7092; A61K 31/192; A61M 37/00; A61F 13/0283

(57) ABSTRACT

Stretchable, occlusive backing layers for transdermal drug delivery systems are disclosed, that maintain occlusivity after stretching. The backing layers are comprised of a stretchable backing material provided with an occlusive coating comprising a styrene-isoprene-styrene block copolymer and tackifier. Also described are transdermal drug delivery systems having such backing layers, including transdermal drug delivery systems for non-steroidal anti-inflammatory drugs (NSAIDs), and methods of making and using such backing layers and transdermal drug delivery systems.

### CROSS-REFERENCE TO RELATED APPLICATIONS

This application claims the benefits under 35 U.S.C. § 119(e) to U.S. provisional application 62/255,700, filed Nov. 16, 2015, the contents of which are incorporated herein by reference in their entirety.

### FIELD

The present invention relates generally to transdermal drug delivery systems, and in particular to stretchable backing layers useful in transdermal drug delivery systems. In specific embodiments, the stretchable backing layers maintain their moisture vapor transition rate properties after stretching. The invention also relates to transdermal drug delivery systems having such backing layers, including transdermal drug delivery systems for non-steroidal anti-inflammatory drugs (NSAIDs), and to methods of making and using such backing layers and transdermal drug delivery systems.

### BACKGROUND

The use of transdermal drug delivery systems, such as transdermal drug delivery patches, to administer an active agent through the skin or mucosa is well known. Such systems typically incorporate the active agent into a carrier composition, such as a polymeric and/or pressure-sensitive adhesive composition, from which the active agent is delivered through the skin or mucosa of the user. Such systems usually are provided with a backing layer that protects other layers and components of the system, and prevents loss of components to the environment during use.

Many factors influence the design and performance of transdermal drug delivery systems, such as the individual drugs themselves, the physical/chemical characteristics of the system's components and the performance/behavior relative to other system components once combined, external/environmental conditions during manufacturing and storage thereafter, the properties of the topical site of application, the desired rate of drug delivery and onset, the drug delivery profile, and the intended duration of delivery. Cost, appearance, size and ease of manufacturing also are important considerations. The properties of the backing layer can influence many aspects of the performance of transdermal drug delivery systems, including pharmacokinetic properties (e.g., the rate and/or duration of drug delivery) and physical properties (e.g., wear properties).

US 2014/0188056 describes transdermal drug delivery systems for NSAIDs that may have an occlusive, flexible, stretchable backing layer comprised of a fabric backing material coated with an occlusive coating. However, the moisture vapor transmission rate (MVTR) of the backing material exemplified in that application may increase after the backing is stretched (e.g., after elongation by 20% or greater) or after storage at 40° C. or 60° C.

Thus, there remains a need for backing layers that maintain desired properties after stretching.

### SUMMARY

Described are stretchable, occlusive backing layers for transdermal drug delivery systems. In some embodiments, the stretchable, occlusive backing layers maintain their moisture vapor transition rate properties after stretching. Also described are transdermal drug delivery systems having such backing layers, and methods of making and using such backing layers and transdermal drug delivery systems.

In specific embodiments, the stretchable, occlusive backing layer comprises a stretchable backing material coated with an occlusive polymer coating comprising a styrene-isoprene-styrene block copolymer (SIS) and tackifier. In some embodiments, the tackifier comprises a hydrogenated hydrocarbon resin (HHR), such as a C5 to C9 HHR. In specific embodiments, the stretchable material is a stretchable cloth material, such as a woven or non-woven cloth material.

In some embodiments, the occlusive polymer coating comprises from 10 to 90%, or from 10 to 70%, by weight HHR, based on the dry weight of the occlusive polymer coating. In some embodiments, the occlusive polymer coating comprises from 10 to 90% by weight SIS, based on the dry weight of the occlusive polymer coating. In some embodiments, the ratio of SIS to HHR in the occlusive polymer coating is from about 20:80 to about 80:20.

In some embodiments, the occlusive polymer coating further comprises a polyisobutylene polymer. In some embodiments, the polyisobutylene polymer is present in an amount of up to 25% by weight of the occlusive polymer coating.

In some embodiments, the occlusive polymer coating is applied to the stretchable backing material at a coat weight of from about 1 mg/cm² to about 15 mg/cm². In some embodiments, the occlusive polymer coating is applied to the stretchable backing material at a coat weight of from about 3.5 mg/cm² to about 11 mg/cm².

In some embodiments, the backing layer has a moisture vapor transmission rate of less than about 60 g/m²/day after stretching to 66% elongation. In some embodiments, the backing layer has a moisture vapor transmission rate of less than about 100 g/m²/day after storage for 6 months at 40° C.

Also provided are transdermal drug delivery system in the form of a flexible, finite system comprising a stretchable, occlusive backing layer as described herein and a drug-containing polymer matrix. In some embodiments, the drug-containing polymer matrix comprises an NSAID, such as flurbiprofen.

Also provided are methods for preparing a stretchable, occlusive backing that exhibits occlusivity after stretching to an elongation of 20% or after storage for 6 months at 40° C., comprising providing a stretchable backing material with an occlusive polymer coating comprising a styrene-isoprene-styrene block copolymer (“SIS”) and tackifier, as described herein. In some embodiments, the stretchable backing material is prepared to exhibit any one or more of the properties set forth above and described in more detail below.

Also provided are methods for the transdermal delivery of a drug, comprising topically applying a transdermal drug delivery system as described herein, comprising a stretchable, occlusive backing layer as described herein, to the skin or mucosa of a subject in need thereof. In some embodiments, the transdermal drug delivery system is topically applied to a joint of a subject in need thereof. In some embodiments, the transdermal drug delivery system comprises a drug-containing polymer matrix comprising an NSAID. In some embodiments, the transdermal drug delivery system comprises a drug-containing polymer matrix comprising flurbiprofen.

Also provided are transdermal drug delivery systems as described herein, comprising a stretchable, occlusive backing layer as described herein, for use in transdermally delivering the drug to a subject in need thereof, or for use in treating pain or inflammation in a subject in need thereof.

Also provided are uses of a stretchable, occlusive backing layer as described herein in the preparation of a medicament for treating pain or inflammation, wherein the medicament is a transdermal drug delivery system comprising the backing layer and a drug-containing polymer matrix comprising a drug, such as an NSAID.

### BRIEF DESCRIPTION OF THE DRAWINGS

FIGS. 1A and 1B show the results of in vitro drug flux studies of flurbiprofen from systems having a PIB coated cloth backing (A) and from systems having an SIS coated cloth backing as described herein (B), after storage at room temperature or 40° C., as compared to drug flux from a commercial flubiprofen patch (20 mg/70 cm² YAKUBAN® Tape, ♦).

### DETAILED DESCRIPTION

Described herein are backing layers for transdermal drug delivery systems. In some embodiments, the backing layers are occlusive, flexible, and/or stretchable. Also described are transdermal drug delivery systems having such backing layers, and methods of making and using such backing layers and transdermal drug delivery systems.

Definitions

Technical and scientific terms used herein have the meanings commonly understood by one of ordinary skill in the art to which the present invention pertains, unless otherwise defined. Reference is made herein to various methodologies known to those of ordinary skill in the art. Publications and other materials setting forth such known methodologies to which reference is made are incorporated herein by reference in their entireties as though set forth in full. Any suitable materials and/or methods known to those of ordinary skill in the art can be utilized in carrying out the present invention. However, specific materials and methods are described. Materials, reagents and the like to which reference is made in the following description and examples are obtainable from commercial sources, unless otherwise noted.

As used herein, the singular forms “a,” “an,” and “the” designate both the singular and the plural, unless expressly stated to designate the singular only.

The term “about” means that the number comprehended is not limited to the exact number set forth, and is intended to encompass values around the stated value while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

The phrase “substantially free” as used herein means that the described composition (e.g., polymer matrix, etc.) comprises less than about 5%, less than about 3%, or less than about 1% by weight, based on the total weight of the composition at issue, of the excluded component(s).

As used herein “subject” denotes any mammal in need of drug therapy, including humans. For example, a subject may be suffering from or at risk of developing a condition that can be treated or prevented with an NSAID (such as pain or inflammation), or may be taking an NSAID for other purposes.

As used herein, the terms “topical” and “topically” mean application to a skin or mucosal surface of a mammal, while the terms “transdermal” and “transdermal” connote passage through the skin or mucosa (including oral, buccal, nasal, rectal and vaginal mucosa), into systemic circulation. Thus, the compositions described herein may be applied topically to a subject to achieve transdermal delivery of an NSAID.

As used herein, the phrases “therapeutically effective amount” and “therapeutic level” mean that drug dosage or plasma concentration in a subject, respectively, that provides the specific pharmacological effect for which the drug is administered in a subject in need of such treatment. It is emphasized that a therapeutically effective amount or therapeutic level of a drug will not always be effective in treating the conditions/diseases described herein, even though such dosage is deemed to be a therapeutically effective amount by those of skill in the art. For convenience only, exemplary dosages, drug delivery amounts, therapeutically effective amounts and therapeutic levels are provided below with reference to adult human subjects. Those skilled in the art can adjust such amounts in accordance with standard practices as needed to treat a specific subject and/or condition/disease.

The transdermal drug delivery systems described herein are in a “flexible, finite form.” As used herein, the phrase “flexible, finite form” means a substantially solid form capable of conforming to a surface with which it comes into contact, and capable of maintaining contact so as to facilitate topical application. Such systems in general are known in the art and commercially available, such as transdermal drug delivery patches.

The compositions comprise a drug-containing polymer matrix that releases the drug, such as an NSAID, upon application to the skin (or any other surface noted above). The compositions in flexible, finite form also include a backing layer in addition to the drug-containing polymer matrix layer. In some embodiments, the compositions in flexible, finite form may include a release liner layer in addition to a drug-containing polymer matrix layer and backing layer.

As used herein, “drug-containing polymer matrix” refers to a polymer composition which contains one or more drugs, such as one or more NSAIDs, and a polymer, such as a pressure-sensitive adhesive polymer or a bioadhesive polymer. A polymer is an “adhesive” or “bioadhesive” if it has the properties of adhesiveness per se. Other polymers can function as an adhesive or bioadhesive by the addition of tackifiers, plasticizers, crosslinking agents or other excipients. Thus, in some embodiments, the polymer optionally comprises tackifiers, plasticizers, crosslinking agents or other additives known in the art.

As used herein, the term “pressure-sensitive adhesive” refers to a viscoelastic material which adheres instantaneously to most substrates with the application of very slight pressure and remains permanently tacky. As noted above, a polymer is a pressure-sensitive adhesive polymer if it has the properties of a pressure-sensitive adhesive per se. Other polymers may function as a pressure-sensitive adhesive by admixture with tackifiers, plasticizers or other additives. The term pressure-sensitive adhesive also includes mixtures of different polymers.

In some embodiments, the polymer matrix is a pressure-sensitive adhesive at room temperature and exhibits desirable physical properties, such as good adherence to skin, ability to be peeled or otherwise removed without substantial trauma to the skin, retention of tack with aging, etc. In some embodiments, the polymer matrix has a glass transition temperature (T_{g}), measured using a differential scanning calorimeter, of between about −70° C. and 0° C.

In some embodiments, the compositions in flexible, finite form are “monolithic” or “monolayer” systems, such that the drug-containing polymer matrix layer is the only polymeric layer present other than the backing layer and the release liner, if present. In such embodiments, the polymer matrix functions as both the drug carrier and the means of affixing the system to the skin or mucosa.

Stretchable Backing Layer

The backing layers described herein are designed to protect other layers and components of the system, and prevent loss of components to the environment during use. In some embodiments, the backing layer is substantially impermeable to the drug(s) and/or other components formulated in the carrier composition, to prevent or minimize loss of drug and/or other components through the backing layer. In some embodiments, the backing layer is stretchable (and, optionally, flexible) and occlusive. As used herein, the term “occlusive” refers backing layers having a limited moisture vapor transmission rate. In specific embodiments, the moisture vapor transmission rate is less than about 300 g/m²/day, less than about 200 g/m²/day, or less than about 100 g/m²/day, including less than 300 g/m²/day, less than 200 g/m²/day, or less than 100 g/m²/day, such as from about 10 to about 100 g/m²/day or from about 20 to about 100 g/m²/day, including from 10 to 100 g/m²/day or 20 to 100 g/m²/day. Stretchable (and, optionally, flexible) and occlusive embodiments are particularly suitable for use on areas of the body that are flexed and/or experience movement, such as joints, while still providing good drug flux. Such a backing layer can be made, for example, by applying an occlusive coating comprising a styrene-isoprene-styrene (SIS) block copolymer and tackifier to a cloth backing material, as described in more detail below and illustrated in the examples.

A stretchable (and, optionally, flexible) and occlusive backing layer as described herein exhibits increased flux as compared to conventional non-occlusive stretchable backing layers (for example, backings comprised of non-woven fabric), which generally exhibit low drug flux because of their relatively low occlusivity and relatively high moisture vapor transmission rates (MVTRs). In some embodiments, a stretchable (and, optionally, flexible) and occlusive backing layer as described herein maintains a low MVTR after stretching (e.g., after elongation by 20% or greater) and/or after storage at 40° C. or 60° C., whereas previously described stretchable occlusive backing layers (such as the polyisobutylene-coated backing layers described in the examples of US 2014/0188056) may exhibit increased MVTRs after stretching. Because an increased MVTR may be associated with decreased drug flux, a stretchable occlusive backing layer that maintains a low MVTR after stretching as described herein also may maintain its drug flux properties after stretching, whereas previously described stretchable occlusive backing layers that exhibit increased MVTRs after stretching may exhibit decreased drug flux after stretching.

As noted above, in some embodiments, a stretchable (and, optionally, flexible) and occlusive backing layer as described herein comprises a stretchable (and, optionally, flexible) backing material provided with an occlusive coating, such as a coating comprising an SIS block copolymer and tackifier.

In some embodiments, the backing material is a stretchable (and, optionally, flexible) cloth material, such as a woven or non-woven cloth material. Stretchable (and, optionally, flexible) cloth materials suitable for use as backing materials for transdermal drug delivery systems are known in the art and available commercially.

SIS polymers suitable for use in a polymer matrix of a transdermal drug delivery system can be used as the SIS component of an occlusive coating as described herein. Such SIS polymers are known in the art and available commercially, such as those sold by Kraton under the KRATON® brand, such as the KRATON® D (SIS) polymers, such as KRATON® D111 KT. KRATON® D (SIS) polymers are block copolymers in which the elastomeric midblock of the molecules is an unsaturated rubber (SIS). Those that have low polystyrene content, such as about 16% to about 24%, are advantageous for creating a softer polymer with a lower modulus suitable for formulating soft, tacky pressure-sensitive adhesives.

Suitable tackifiers include rosin esters, rosin resins, aliphatic hydrocarbon resins, aromatic hydrocarbon resins, terpene resins, polybutene, and hydrogenated polybutene. In specific embodiments, the tackifier is a C5 to C9 hydrogenated hydrocarbon resin (HHR), such as REGALITE® R1090, R1100, or R1125 by Eastman, or ARKON® P-70, P-80, P-90, P-100, P-1 15, or P-125 by Arakawa Chemical. The occlusive coating may comprise from about 10% to about 70% by weight tackifier (such as HHR), based on the dry weight of the occlusive coating, including from 10% to 70% by weight tackifier, including about 10%, 15%, 20%, 25%, 30%, 35%, 40%, 45%, 50%, 55%, 60%, 65%, or 70% by weight tackifier, including 10%, 15%, 20%, 25%, 30%, 35%, 40%, 45%, 50%, 55%, 60%, 65%, or 70% by weight tackifier, based on the dry weight of the occlusive coating.

In some embodiments, the occlusive coating further comprises another polymer, such as a polyisobutylene (PIB) polymer. A PIB polymer suitable for use in a polymer matrix of a transdermal drug delivery system can be used as the PIB component of an occlusive coating as described herein. Such PIB polymers are known in the art and available commercially, such as those sold by BASF under the OPPANOL® B brand, which is a series of medium and high molecular weight PIB polymers having a weight-average molecular weight (Mw) between 40,000 and 4,000,000, and include OPPANOL® B100 and OPPANOL® B11SFN. In some embodiments, the PIB polymer is PIB513, which is an adhesive solution containing 6.29% OPPANOL® B100 (MW 1,110,000), 37.39% OPPANOL® B11SFN (MW 46,000) and 55.92% toluene. In other embodiments, the PIB polymer comprises OPPANOL ® B100 and OPPANOL® B11SFN in any suitable ratio, including a ratio of 35:65 by weight.

When the occlusive coating includes an additional polymer in addition to the SIS block copolymer and tackifier, such as a PIB polymer, the additional polymer may be present in an amount of from about 1% to about 25% by weight of the occlusive coating, including an amount of about 1%, about 5%, about 10%, about 15%, about 20%, or about 25% by dry weight of the occlusive coating. In specific embodiments, the coating includes at least about 75% by weight of the SIS/HHR component, including at least 75% by weight SIS/HHR component, including about 75% to 100% by weight SIS/HHR component, such as about 75%, 80%, 85%, 90%, 95% or 100% by weight SIS/HHR component, including 75%, 80%, 85%, 90%, 95% or 100% by weight SIS/HHR component.

The moisture vapor transmission rate of a stretchable backing layer as described herein can be controlled, for example, by controlling the specific components of the occlusive coating and/or the thickness of the occlusive coating, as illustrated in the examples below. For example, increasing the ratio of tackifier to SIS block copolymer in the occlusive coating generally results in a backing with a lower MVTR, and increasing the thickness of the occlusive coating generally results in a backing with a lower MVTR. In some embodiments, the ratio of SIS block copolymer to tackifier (by weight) in the occlusive coating is from about 10:90 to 90:10, including from about 20:80 to about 70:30, including 10;90, 20:80, 40:60, 50:50, 60:40, 70:30, 80:20, and 90:10 SIS block copolymer to tackifier. In some embodiments, the occlusive coating is applied to the backing material at a thickness of from about 2 mg/cm² to about 15 mg/cm², including a thickness of about 2, 3, 3.5, 5, 7, 9, 11, 13 or 15 mg/cm².

In some embodiments, a stretchable (and, optionally, flexible) and occlusive backing layer as described herein can be used to manufacture a system with a moisture vapor transmission rate that is the same as or even lower than a comparable system with a plastic backing, as illustrated in the examples below and/or that maintains its occlusivity (e.g., its low MVTR) after stretching (e.g., after elongation by 20% or greater) and/or after storage at 40° C. or 60° C. In some embodiments, the stretchable backing has a MVTR of less than about less than about 300 g/m²/day, less than about 200 g/m²/day, or less than about 100 g/m²/day, including less than 300 g/m²/day, less than 200 g/m²/day, or less than 100 g/m²/day, such as from about 10 to about 100 g/m²/day or from about 20 to about 100 g/m²/day, including from 10 to 100 g/m²/day or 20 to 100 g/m²/day. In some embodiments, the backing layer exhibits a moisture vapor transmission rate of less than about 100 g/m²/day after stretching to 66% elongation. In some embodiments, the backing layer exhibits a moisture vapor transmission rate of less than about 60 g/m²/day after stretching to 66% elongation. In some embodiments, the backing layer exhibits a moisture vapor transmission rate of less than about 100 g/m²/day after storage for 6 months at 40° C. MVTR can be measured by standard procedures, e.g., using cups designated for MVTR evaluation. In a typical protocol (based on ASTM E96), MVTR cups are loaded with calcium chloride, weighed and then sealed with the backing material to be tested. The cups are placed in a humid chamber set to 40° C./100% RH, and a 24-hour test is run to assess how much moisture passes through the backing material from the humid atmosphere into the cups.

Also provided are methods for preparing a stretchable, occlusive backing that exhibits occlusivity after stretching to an elongation of 20%, or after storage for 6 months at 40° C., comprising providing a stretchable backing material in accordance with any of the embodiments described above with an occlusive polymer coating in accordance with any of the embodiments described above. The coating can be prepared by any suitable method, including by blending the coating components in a vessel. The coating can be applied to the backing material by any suitable method, such as by using a coating apparatus typically used in the preparation of transdermal drug delivery systems.

Although the stretchable (and, optionally, flexible) and occlusive backing layer is discussed and illustrated herein below with reference to flexible, finite systems for the transdermal delivery of NSAIDs, it can be used as a backing layer for any flexible, finite transdermal drug delivery system (e.g., for any transdermal drug patch). Indeed, as discussed above, a stretchable (and, optionally, flexible) and occlusive backing layer is particularly useful for systems that may be applied to areas of the body that are flexed and/or experience movement, such as joints (e.g., knees, elbows, wrists, ankles, fingers, and toes), while also providing good drug flux, and so may be useful for systems formulated with any active agent.

Polymer Matrix

In accordance with some embodiments, the compositions described herein comprise a polymer matrix that comprises, consists essentially of, or consists of, an NSAID and/or pharmaceutically acceptable salt(s) thereof and a silicone polymer, an acrylic polymer and/or an acrylic block copolymer and, optionally, an SIS copolymer. In this context, the phrase “consists essentially of” means that the polymer matrix is substantially free of other polymer components (e.g., substantially free of polymers other than silicone polymer(s), acrylic polymer(s), and styrene-isoprene-styrene block copolymer(s) and skin penetration enhancers, although it may include other excipients known to be useful in transdermal compositions (such as tackifiers, plasticizers, crosslinking agents or other excipients known in the art) as long as those other excipients do not degrade the physical and/or pharmacokinetic properties of the compositions to pharmaceutically unacceptable levels. In accordance with some embodiments, the compositions described herein comprise a polymer matrix that comprises, consists essentially of, or consists of, an NSAID and/or pharmaceutically acceptable salt(s) thereof a silicone polymer, an acrylic polymer and/or an acrylic block copolymer and, optionally, an SIS block copolymer and, optionally, one or more skin penetration enhacers.

NSAID

NSAIDs are known in the art and include ibuprofen, dexibuprofen, naproxen, fenoprofen, ketoprofen, dexketoprofen, flurbiprofen, oxaprozin, loxoprofen, indomethacin, tolmetin, sulindac, etodolac, ketorolac, diclofenac, nabumetone, piroxicam, meloxicam, tenoxicam, droxicam, lornoxicam, isoxicam, mefenamic acid, meclofenamic acid, flufenamic acid, tolfenamic acid, niflumic acid, aspirin, diflunisal, and salsalate.

In specific embodiments, the NSAID is flurbiprofen. Flurbiprofen has anti-inflammatory, analgesic and antipyretic properties. It is used, for example, to treat rheumatoid arthritis, osteoarthritis, and to prevent miosis during ocular surgery.

The compositions described herein may be formulated with an NSAID in its free acid form, or as any pharmaceutically acceptable ester thereof, or any combinations thereof. Exemplary suitable pharmaceutically acceptable salts are salts of weak inorganic and organic acids, and quaternary ammonium salts. These include without limitation, salts with acids such as sulfuric, phosphoric, hydrochloric, hydrobromic, hydriodic, sulfamic, citric, lactic, maleic, malic, succinic, tartaric, cinnamic, acetic, benzoic, gluconic, or ascorbic acid, or quaternary ammonium salts with organic esters of sulfuric, hydrohalic, or aromatic sulfonic acids, such as methyl chloride, methyl bromide, ethyl chloride, propyl chloride, butyl chloride, isobutyl chloride, benzylchloride, benzyl bromide, phenethyl bromide, naphthymethyl chloride, dimethyl sulfate, methyl benzenesulfonate, ethyl toluenesulfonate, ethylene chlorohydrin, propylene chlorobydrin, allyl bromide, methylallyl bromide or crotyl bromide esters.

The compositions described herein include a therapeutically effective amount of NSAID or pharmaceutically acceptable salt(s) thereof. Generally, the amount of NSAID is from about 0.1% to about 50%, including from about 1% to about 20%, such as from about 1% to about 10% by weight, such as about 1, about 2, about 3, about 4, about 5, about 6, about 7, about 8, about 9 or about 10% by weight, based on the total dry weight of the polymer matrix. In specific embodiments, the polymer matrix comprises about 3-5% by weight NSAID, based on the total dry weight of the polymer matrix, such as about 3% or about 5% by weight NSAID, based on the total dry weight of the polymer matrix.

When the compositions are used for local effect, they may include from about 20 to about 35 mg of NSAID (such as flurbiprofen). The compositions have specific advantages when used for local effect, e.g., to treat conditions at or near the application site. In addition to avoiding the gastrointestinal tract and associated side effects, the compositions are able to deliver a high dose of NSAID directly to the site to be treated, while reducing or minimizing undesired systemic effects.

Silicone Polymers

As noted above, in some embodiments the polymer matrix comprises one or more silicone polymers, such as one or more pressure-sensitive adhesive silicone polymers. Silicone polymers suitable for use in polymer matrix compositions are known.

The term “silicone-based” polymer is used interchangeably with the terms silicon polymers, siloxane, polysiloxane, and silicones as used herein and as known in the art. A suitable silicone-based polymer may also be a pressure-sensitive adhesive. Thus, in some embodiments, the silicone-based polymer is an adhesive polymer. In other embodiments, the silicone-based polymer functions as an adhesive by the addition of tackifiers, plasticizers, crosslinking agents, or other additives.

Suitable polysiloxanes include silicone pressure-sensitive adhesives which are based on two major components: (i) a polymer or gum and (ii) a tackifying resin. A polysiloxane adhesive can be prepared by cross-linking a gum, typically a high molecular weight polydiorganosiloxane, with a resin, to produce a three-dimensional silicate structure, via a condensation reaction in an appropriate organic, volatile solvent, such as ethyl acetate or heptane. The ratio of resin to polymer can be adjusted in order to modify the physical properties of polysiloxane adhesives. Sobieski, et al., “Silicone Pressure Sensitive Adhesives,” Handbook of Pressure-Sensitive Adhesive Technology, 2nd ed., pp. 508-517 (D. Satas, ed.), Van Nostrand Reinhold, New York (1989).

Exemplary silicone-based polymers are adhesives (e.g., capable of sticking to the site of topical application), including pressure-sensitive adhesives. Illustrative examples of silicone-based polymers having reduced silanol concentrations include silicone-based adhesives (and capped polysiloxane adhesives) such as those described in U.S. Pat. No. Re. 35,474 and U.S. Pat. No. 6,337,086, which are incorporated herein by reference in their entireties, and which are commercially available from Dow Corning Corporation (Dow Corning Corporation, Medical Products, Midland, Mich.) as BIO-PSA® 7-4100, -4200 and -4300 product series, and non-sensitizing, pressure-sensitive adhesives produced with compatible organic volatile solvents (such as ethyl acetate or heptane) and available commercially under their BIO-PSA® 7-4400 series, -4200 series, such as -4202 and -42-3, and the -4500 series, such as -4502, such as -4503, and -4600 series.

Further details and examples of silicone pressure-sensitive adhesives which are useful in the polymer matrices and compositions and methods described herein are mentioned in the following U.S. Pat. Nos.: 4,591,622; 4,584,355; 4,585,836; and 4,655,767, which are all expressly incorporated by reference herein in their entireties. It should also be understood that silicone fluids are also contemplated for use in the polymer matrices and methods described herein.

### Acrylic Polymers

As noted above, in some embodiments the polymer matrix comprises one or more acrylic polymers, such as one or more pressure-sensitive adhesive acrylic polymers. Acrylic polymers suitable for use in polymer matrix compositions are known.

The term “acrylic polymer” is used here as in the art interchangeably with “polyacrylate,” “polyacrylic polymer,” and “acrylic adhesive.” The acrylic-based polymers can be any of the homopolymers, copolymers, terpolymers, and the like of various acrylic acids or esters. In some embodiments, the acrylic-based polymers are adhesive polymers. In other embodiments, the acrylic-based polymers function as an adhesive by the addition of tackifiers, plasticizers, crosslinking agents or other additives.

The acrylic polymer can include copolymers, terpolymers and multipolymers. For example, the acrylic polymer can be any of the homopolymers, copolymers, terpolymers, and the like of various acrylic acids. In some embodiments, the acrylic polymer constitutes from about 2% to about 95% by weight of the polymer content of the polymer matrix, including about 3% to about 90% and about 5% to about 85%, such as 2% to 95%, 3% to 90% and 5% to 85%. In some embodiments, the amount and type of acrylic polymer is dependent on the type and amount of therapeutically active agents used.

Acrylic polymers useful in practicing the invention include polymers of one or more monomers of acrylic acids and other copolymerizable monomers. The acrylic polymers also include copolymers of alkyl acrylates and/or methacrylates and/or copolymerizable secondary monomers or monomers with functional groups. Combinations of acrylic-based polymers based on their functional groups is also contemplated. Acrylic-based polymers having functional groups include copolymers and terpolymers which contain, in addition to nonfunctional monomer units, further monomer units having free functional groups. The monomers can be monofunctional or polyfunctional. By varying the amount of each type of monomer added, the cohesive properties of the resulting acrylic polymer can be changed as is known in the art. In some embodiments, the acrylic polymer is composed of at least 50% by weight of an acrylate or alkyl acrylate monomer, from 0 to 20% of a functional monomer copolymerizable with the acrylate, and from 0 to 40% of other monomers.

Acrylate monomers which can be used include acrylic acid and methacrylic acid and alkyl acrylic or methacrylic esters such as methyl acrylate, ethyl acrylate, propyl acrylate, amyl acrylate, butyl acrylate, butyl methacrylate, hexyl acrylate, methyl methacrylate, hexyl methacrylate, heptyl acrylate, octyl acrylate, nonyl acrylate, 2-ethylbutyl acrylate, 2-ethylbutyl methacrylate, isooctyl acrylate, isooctyl methacrylate, 2-ethylhexyl acrylate, 2-ethylhexyl methacrylate, decyl acrylate, decyl methacrylate, dodecyl acrylate, dodecyl methacrylate, tridecyl acrylate, tridecyl methacrylate, glycidyl acrylate, and corresponding methacrylic esters.

Non-functional acrylic-based polymers can include any acrylic based polymer having no or substantially no free functional groups.

Functional monomers, copolymerizable with the above alkyl acrylates or methacrylates, which can be used include acrylic acid, methacrylic acid, maleic acid, maleic anhydride, hydroxyethyl acrylate, hydroxypropyl acrylate, acrylamide, dimethylacrylamide, acrylonitrile, dimethylaminoethyl acrylate, dimethylaminoethyl methacrylate, tert-butylaminoethyl acrylate, tert-butylaminoethyl methacrylate, methoxyethyl acrylate and methoxyethyl methacrylate.

As used herein, “functional monomers or groups,” are monomer units typically in acrylic-based polymers which have reactive chemical groups which modify the acrylic-based polymers directly or which provide sites for further reactions. Examples of functional groups include carboxyl, epoxy, hydroxyl, sulfoxyl, and amino groups. Acrylic-based polymers having functional groups contain, in addition to the nonfunctional monomer units described above, further monomer units having free functional groups. The monomers can be monofunctional or polyfunctional. These functional groups include carboxyl groups, hydroxy groups, amino groups, amido groups, epoxy groups, etc. Typical carboxyl functional monomers include acrylic acid, methacrylic acid, itaconic acid, maleic acid, and crotonic acid. Typical hydroxy functional monomers include 2-hydroxyethyl methacrylate, 2-hydroxyethyl acrylate, hydroxymethyl acrylate, hydroxymethyl methacrylate, hydroxyethyl acrylate, hydroxyethyl methacrylate, hydroxypropyl acrylate, hydroxypropyl methacrylate, hydroxybutyl acrylate, hydroxybutyl methacrylate, hydroxyamyl acrylate, hydroxyamyl methacrylate, hydroxyhexyl acrylate, hydroxyhexyl methacrylate. As noted above, in some embodiments, the acrylic polymer does not include such functional groups. In other embodiments, the acrylic polymer does not include hydoxy functional groups.

In accordance with specific embodiments, the polymer matrix comprises or consists of one or more non acid-functional acrylic polymers as the polymer component. Non acid-functional acrylic polymers include those formed from acrylic esters copolymerized with other monomers that do not include acid-functional groups. Non acid-functional acrylic polymers include homopolymers, copolymers, terpolymers, etc., of acrylic acids and esters. As used herein, “non acid-functional acrylic polymer” includes polymers that include monomers that have one or more amide groups. In specific embodiments, the non acid-functional acrylic polymer includes methacrylate monomers and 2-ethylhexyl acrylate monomers. In specific embodiments the non acid-functional acrylic polymer includes methacrylate monomers, 2-ethylhexyl acrylate monomers, and amide-group containing monomers.

In some embodiments, the acrylic polymer component of the polymer matrix consists of a single acrylic polymer. In other embodiments, the acrylic polymer component of the polymer matrix comprises a blend of a first acrylic polymer and a second acrylic polymer, and optionally includes additional (e.g., a third or more) acrylic polymers.

When the acrylic polymer component includes more than one acrylic polymer, the polymers can be present in any ratio that results in a product with satisfactory physical and pharmacokinetic properties. For example, the acrylic polymer component can include from 0-100% of a first acrylic polymer and from 100-0% of a second acrylic polymer, based on the total dry weight of the acrylic component, including about 10 to about 90%, about 15- about 85%, about 20 to about 80%, about 25 to about 75%, about 33 to about 66%, and about 50% of the first acrylic polymer, and the balance being the second (or third, etc.) acrylic polymer(s). In specific embodiments, the acrylic polymer component includes about 80% of a first acrylic polymer and about 20% of a second acrylic polymer, based on the total polymer content.

Suitable acrylic polymers also include pressure-sensitive adhesives which are commercially available, such as the acrylic-based adhesives sold under the trademarks DURO-TAK®, such as 900A or 87-9900, and GELVA®, such as 3087 and 3235, by Henkel Corporation, Bridgewater, N.J. Other suitable acrylic polymers are known in the art.

Further details and examples of similarly suitable acrylic adhesives which are suitable in the practice of the invention are described in Satas, “Acrylic Adhesives,” Handbook of Pressure-Sensitive Adhesive Technology, 2nd ed., pp. 396-456 (D. Satas, ed.), Van Nostrand Reinhold, New York (1989); “Acrylic and Methacrylic Ester Polymers,” Polymer Science and Engineering, Vol. 1, 2nd ed., pp 234-268, John Wiley & Sons, (1984); U.S. Patent No. 4,390,520; and U.S. Pat. No. 4,994,267, all of which are expressly incorporated by reference in their entireties.

### Acrylic Block Copolymers

As noted above, in some embodiments the polymer matrix comprises one or more acrylic block copolymers, such as one or more pressure-sensitive adhesive acrylic block copolymers, including conjugates of a non-functional acrylic pressure-sensitive adhesive (such as any described above) and silicone fluid polydimethylsiloxane or trimethylsiloxysilane moieties. Suitable acrylic block copolymers are available commercially, such as from Henkel (e.g., Henkel 14700-14 or DURO-TAK® 87-9900).

### Other Polymers

As noted above, in some embodiments the polymer matrix comprises one or more rubber-based polymers, such as one or more rubber-based pressure-sensitive adhesives, such as natural or synthetic polyisoprene, polybutylene, polyisobutylene, styrene-butadiene polymers, SIS copolymers, hydrocarbon polymers, such as butyl rubber, halogen-containing polymers, such as polyacrylic-nitrile, polytetrafluoroethylene, polyvinylchloride, polyvinylidene chloride, and polychlorodiene, and other copolymers thereof. In specific embodiments, the polymer matrix comprises one or more SIS block copolymers.

As noted above, in some embodiments, the polymer matrices of the compositions described herein consist essentially of the NSAID or pharmaceutically acceptable salt(s) thereof and one or more of the polymer(s) described above, although such compositions may include other non-polymer components that do not degrade the physical and/or pharmacokinetic properties of the compositions to pharmaceutically unacceptable levels, such as one or more penetration enhancers, as discussed in more detail below.

### Penetration Enhancers

As noted above, in some embodiments, the polymer matrices of the compositions described herein further comprise one or more penetration enhancers. A “penetration enhancer” is an agent known to accelerate the delivery of the drug through the skin. These agents also have been referred to as accelerants, adjuvants, and sorption promoters, and are collectively referred to herein as “enhancers.” This class of agents includes those with diverse mechanisms of action, including those which have the function of improving percutaneous absorption, for example, by changing the ability of the stratum corneum to retain moisture, softening the skin, improving the skin's permeability, acting as penetration assistants or hair-follicle openers or changing the state of the skin including the boundary layer. In specific embodiments the enhancer(s) serve to both enhance penetration of the NSAID through the stratum corneum and retain the NSAID at a site local to administration.

Illustrative penetration enhancers include but are not limited to polyhydric alcohols such as dipropylene glycol, propylene glycol, and polyethylene glycol; oils such as olive oil, squalene, and lanolin; fatty ethers such as cetyl ether and oleyl ether; fatty acid esters such as isopropyl myristate; glycerol mono-, di- and tri-esters of fatty acids, such as glycerol monooleate; urea and urea derivatives such as allantoin which affect the ability of keratin to retain moisture; polar solvents such as dimethyidecylphosphoxide, methyloctylsulfoxide, dimethyllaurylamide, dodecylpyrrolidone, isosorbitol, dimethylacetonide, dimethylsulfoxide, decylmethylsulfoxide, and dimethylformamide which affect keratin permeability; salicylic acid which softens the keratin; amino acids which are penetration assistants; benzyl nicotinate which is a hair follicle opener; and higher molecular weight aliphatic surfactants such as lauryl sulfate salts which change the surface state of the skin and drugs administered. Other agents include oleic and linoleic acids, ascorbic acid, panthenol, butylated hydroxytoluene, tocopherol, tocopheryl acetate, tocopheryl linoleate, propyl oleate, and isopropyl palmitate.

In some embodiments, a combination of enhancers is used. For example, a dual enhancer system comprising isopropyl myristate and oleic acid may be particularly useful for formulating NSAIDs, such as flurbiprofen.

Generally speaking, the polymer matrices may include NSAID in an amount from about 1% to about 50%, including from about 1% to about 10%, such as from about 1% to about 5%, including about 1%, about 2%, about 3%, about 4% about 5%, about 6%, about 7%, about 8%, about 9%, or about 10% by weight, based on the total dry weight of the polymer matrix, including about 3-5%, about 3% and about 5%.

Generally speaking, the silicone pressure-sensitive adhesive(s), if present, may be present in a range from about 1% to about 99%, including from about 50% to about 99%, such as from about 80% to about 99%, including from about 90% to about 99%, including about 80%, about 81%, about 82% about 83%, about 84%, about 85%, about 86%, about 87%, about 88%, about 89%, about 90%, about 91%, about 92% about 93%, about 94%, about 95%, about 96%, about 97%, about 98%, or about 99%, by weight, based on the total dry weight of the polymer matrix.

Generally speaking, the acrylic polymer(s), if present, may be present in a range from about 1% to about 50%, including from about 1% to about 20%, such as from about 1% to about 10%, including about 2%, about 3%, about 4% about 5%, about 6%, about 7%, about 8%, about 9%, or about 10%, by weight, based on the total dry weight of the polymer matrix.

Generally speaking, the acrylic block copolymer(s), if present, may be present in a range from about 1% to about 50%, including from about 1% to about 20%, such as from about 1% to about 10%, including about 2%, about 3%, about 4% about 5%, about 6%, about 7%, about 8%, about 9%, or about 10%, by weight, based on the total dry weight of the polymer matrix.

Generally speaking, the other polymer(s) (such as, for example, styrene-isoprene-styrene block copolymer(s)), if present, may be present in a range from about 0.1% to about 50%, including from about 0.1% to about 10%, such as from about 0.1% to about 5%, including about 0.2%, about 0.3%, about 0.4%, about 0.5%, about 0.6%, about 0.7%, about 0.8%, about 0.9%, about 1.0%, about 2%, about 3%, about 4%, or about 5%, by weight, based on the total dry weight of the polymer matrix.

Generally speaking, the penetration enhancer(s), if present, each may be present in an amount from about 0.1% to about 10%, such as from about 0.1% to about 5%, including about 0.2%, about 0.4%, about 0.6%, about 0.8%, about 1.0%, about 2%, about 3%, about 4%, about 5%, about 6%, about 7%, about 8%, about 9%, or about 10%, by weight, based on the total dry weight of the polymer matrix. In embodiments using more than one enhancer, each may be present in any amount described herein (e.g., from about 0.1% to about 10%) or the total amount of enhancers may be within the amounts described herein (about 0.1% to about 10%).

While not wanting to be bound by any theory it is believed that the polymer blends described herein balance competing goals and properties of drug solubility and drug delivery. For example, a silicone polymer-based system may have a solubility for the NSAID (such as flurbiprofen) that is so low (e.g., 1%) that it is difficult to formulate a sufficient amount of NSAID to achieve delivery over an extended time period. On the other hand, an acrylic polymer-based system may have a solubility for the NSAID (such as flurbiprofen) that is so high (e.g., 15%) that very high drug loading is required to achieve drug flux out of the system. The inventors have discovered that the polymer blends described herein, comprising a silicone-based polymer and an acrylic polymer and/or an acrylic block copolymer and, optionally, an SIS block copolymer, balances these competing properties and achieves good drug flux without requiring high drug loading.

Release Liner

The compositions in flexible, finite form may further comprise a release liner, typically located adjacent the opposite face of the system as compared to the backing layer. When present, the release liner is removed from the system prior to use to expose the polymer matrix layer prior to topical application. Materials suitable for use as release liners are well-known known in the art and commercially available, such as polyester release liners, including coated polyester release liners.

Methods of Manufacture

The compositions described herein can be prepared by methods known in the art. As one step, the polymer matrices described herein can be prepared by methods known in the art, such as blending (mixing) the polymer components in powder or liquid form with an appropriate amount of drug in the presence of an appropriate solvent, such as a volatile organic solvent, optionally with other excipients. To form a final product, the drug/polymer/solvent mixture may be cast onto a release liner (optionally, at ambient temperature and pressure) followed by evaporation of the volatile solvent(s), for example, at room temperature, slightly elevated temperature, or by a heating/drying step, to form the drug-containing polymer matrix on a release liner. A backing layer as described herein may be applied to form a final product.

An exemplary general method for preparing a unit final product of a composition as described herein in a flexible, finite form, is as follows:

1. Appropriate amounts of one or more polymers, solvent(s) and/or co-solvent(s), and optional excipient(s) are combined and thoroughly mixed together in a vessel.

2. The NSAID is added to the mixture and agitation is carried out until the drug is uniformly mixed therein.

3. The composition is transferred to a coating operation where it is coated onto a release liner at a controlled specified thickness. The coated composition is then passed through an oven in order to drive off all volatile processing solvents.

4. The composition coated on the release liner is then brought into contact with a previously prepared laminated backing layer and wound into rolls.

5. Appropriate size and shape delivery systems are die-cut from the roll material and then pouched.
As set forth above, a stretchable (and, optionally, flexible) occlusive backing layer can be prepared by applying an occlusive coating as described herein to, for example, a fabric backing material.

The order of steps, the amount of the ingredients, and the amount and time of agitation or mixing may be important process variables which will depend on the specific polymers, active agents, solvents and/or cosolvents, and optional excipients used in the composition, but these factors can be adjusted by those skilled in the art. The order in which each method step is performed can be changed if needed without detracting from the invention.

In accordance with any of the embodiments of compositions described herein, the size of the final product is, in some embodiments, in the range of from about 2 cm² to about 140 cm², including 5 cm², 10 cm², 20 cm², 25 cm², 30 cm², 40 cm², 50 cm², 60 cm², 70 cm², 75 cm², 80 cm², 90 cm², 100 cm², 110 cm², 120 cm², 130 cm², and 140 cm².

Methods of Use

The compositions described herein are useful in methods for the transdermal delivery of an NSAID, including in methods for treating local pain, including chronic or persistent pain, such as may be associated with arthritis, such as rheumatoid arthritis or osteoarthritis. In such embodiments, a composition comprising a therapeutically effective amount of an NSAID, such as flurbiprofen, as described herein is topically applied to a subject in need thereof.

In some embodiments, the compositions achieve transdermal delivery of NSAID over a period of time of at least about 8 hours, including a period of time of at least about 8 hours to at least about 12 hours, at least about 24 hours, or longer.

The compositions described herein achieve a transdermal flux of NSAID (and/or one or more pharmaceutically acceptable salt(s) thereof) that is sufficient to have a therapeutic effect. As used herein, “flux” (also called “permeation rate”) is defined as the absorption of a drug through skin or mucosal tissue, and is described by Fick's first law of diffusion:

J=−D (dCm/dx)

where J is the flux in g/cm²/sec, D is the diffusion coefficient of the drug through the skin or mucosa in cm²/sec and dCm/dx is the concentration gradient of the drug across the skin or mucosa.

The following specific examples are included as illustrative of the compositions described herein. These examples are in no way intended to limit the scope of the invention. Other aspects of the invention will be apparent to those skilled in the art to which the invention pertains.

### EXAMPLE 1

Stretchable, flexible, occlusive backing layers were prepared by applying various coatings comprised of SIS block copolymer (KRATON® D1111) and HHR tackifier (ARKON® P-100) to a cloth backing material, and the moisture vapor transmission rate (MVTR) of the backing layers were assessed.

MVTR is measured by standard procedures, e.g., using cups designated for MVTR evaluation. The cups are loaded with calcium chloride, weighed and then sealed by the backing material being tested. The cups are placed in a humid chamber set to 40° C./100% RH. A 24-hour test is run to assess how much moisture passed through the backing material from the humid atmosphere into the cups.

The results reported in the table below show that increasing the ratio of SIS block copolymer to HHR decreased the occlusivity (increased the MVTR) of the backing layer:

| Sample (5 mg/cm² occlusive coating | MVTR |
|---|---|
| on cloth backing) | (g/m²/day) |
| 40% SIS/60% HHR | 29.66 |
| 50% SIS/50% HHR | 43.97 |
| 60% SIS/40% HHR | 57.39 |
| 70% SIS/30% HHR | 69.76 |

The results reported in the table below show that increasing the thickness of the occlusive coating on the backing material increased the occlusivity (decreased the MVTR) of the backing layer.

| Sample (40% SIS/60% HHR coating | MVTR |
|---|---|
| on cloth backing) | (g/m²/day) |
| 5 mg/cm² | 29.66 |
| 7 mg/cm² | 19.14 |
| 9 mg/cm² | 16.34 |
| 11 mg/cm² | 12.76 |
| Sample (70% SIS/30% HHR coating | MVTR |
| on cloth backing) | (g/m²/day) |
| 5 mg/cm² | 69.76 |
| 7 mg/cm² | 57.74 |
| 9 mg/cm² | 41.76 |
| 11 mg/cm² | 34.50 |

### EXAMPLE 2

The effect of stretching on the MVTR of stretchable, flexible, occlusive backing layers as described herein was assessed and compared to that of a flexible, occlusive backing layer as described in US 2014/0188056, having a PIB-coated backing layer.

| | | MVTR |
|---|---|---|
| Sample | % Elongation | (g/m²/day) |
| PIB coating (4 mg/cm²) | 0 | 12.33 |
| | 10 | 12.91 |
| | 20 | 738.25 |
| | 30 | 1477.83 |
| | 40 | 1832.23 |
| | 66 | 2277.63 |
| 40% SIS/60% HHR | 0 | N/D |
| (3.5 mg/cm²) | 66 | 57.14 |
| 40% SIS/60% HHR | 0 | 29.66 |
| (5 mg/cm²) | 66 | 34.70 |
| 40% SIS/60% HHR | 0 | 19.14 |
| (7 mg/cm²) | 66 | 38.32 |
| 40% SIS/60% HHR | 0 | 16.34 |
| (9 mg/cm²) | 66 | 26.76 |
| 40% SIS/60% HHR | 0 | 12.76 |
| (11 mg/cm²) | 66 | N/D |

### EXAMPLE 3

Stretchable, flexible, occlusive backing layers were prepared by applying various coatings comprised of SIS block copolymer (KRATON® D1111), HHR tackifier (ARKON® P-100)and PIB polymer (35:65 OPPANOL® B100: B11SFN) to a cloth backing material, and the moisture vapor transmission rate (MVTR) of the backing layers were assessed.

The results show that including PIB polymer in the occlusive coating increased the occlusivity (decreases the MVTR) of the backing layer, and that MVTR resistance to stretching was achieved by also including SIS block copolymer and HHR tackifier in the occlusive coating.

| Sample (5 mg/cm² occlusive coating | | MVTR |
|---|---|---|
| on cloth backing) | % Elongation | (g/m²/day) |
| 90% [40% SIS/60% HHR]/10% PIB | 0 | 26.60 |
| | 20 | 30.40 |
| | 40 | 75.64 |
| 35% SIS/60% HHR/5% PIB | 0 | 44.99 |
| | 20 | 22.77 |
| | 40 | 33.61 |
| 15% SIS/60% HHR/25% PIB | 0 | 3.25 |
| | 20 | 12.47 |
| | 40 | 23.31 |

### EXAMPLE 4

Transdermal drug delivery systems comprising a polymer matrix comprising flurbiprofen and different backing layers were prepared.

The following polymer matrix was used for each system:

| | |
|---|---|
| Flurbiprofen: | 5.00% |
| DURO-TAK ® 87-9900: | 4.4% |
| BIO-PSA ® 4502: | 83.6% |
| Isopropyl Myristate: | 2.0% |
| Oleic Acid | 2.0% |
| Povidone 30 | 3.0% |

The following backing layers were used: (i) PIB coated cloth backing; 40% SIS/60% HHR coated cloth backing; YAKUBAN® Tape (Flurbiprofen commercial patch by Tokuhon Corporation, Minato-ku, Tokyo).

The systems were stored under various conditions and MVTR was assessed. These results show that backing layers with occlusive coatings formulated with SIS block copolymer and HHR tackifier were more resistant to increases in MVTR after storage under accelerated conditions as compared to backing layers with occlusive coatings formulated with only PIB polymer. This means that the backing layers as described herein maintained good occlusivity (relatively low MVTRs) after storage under accelerated conditions, indicating that they would maintain acceptable drug flux after storage under accelerated conditions.

| Backing | Storage Conditions | MVTR (g/m²/day) |
|---|---|---|
| PIB (4 mg/cm²) | RT, 1 M | 24.83 |
| | RT, 3 M | 30.32 |
| | RT, 6 M | 35.56 |
| | 40° C., 1 M | 30.43 |
| | 40° C., 3 M | 68.41 |
| | 40° C., 6 M | 1410.73 |
| | 60° C., 1 M | 152.59 |
| PIB (5 mg/cm²) | RT, 1 M | 19.50 |
| | RT, 3 M | 25.78 |
| | RT, 6 M | 27.38 |
| | 40° C., 1 M | 30.43 |
| | 40° C., 3 M | 63.30 |
| | 40° C., 6 M | 252.43 |
| | 60° C., 1 M | 64.27 |
| 40% SIS/60% HHR | Initial | 55.09 |
| (5 mg/cm²) | 40° C., 3 M | 60.15 |
| | 40° C., 5 M | 88.24 |
| | 60° C., 1 M | 47.24 |
| 40% SIS/60% HHR | Initial | 38.3 |
| (7 mg/cm²) | 40° C., 3 M | 44.13 |
| | 40° C., 5 M | 56.82 |
| | 60° C., 1 M | 37.69 |
| 40% SIS/60% HHR | Initial | 26.26 |
| (9 mg/cm²) | 40° C., 3 M | 39.15 |
| | 40° C., 5 M | 38.69 |
| | 60° C., 1 M | 37.69 |

RT = Room temperature

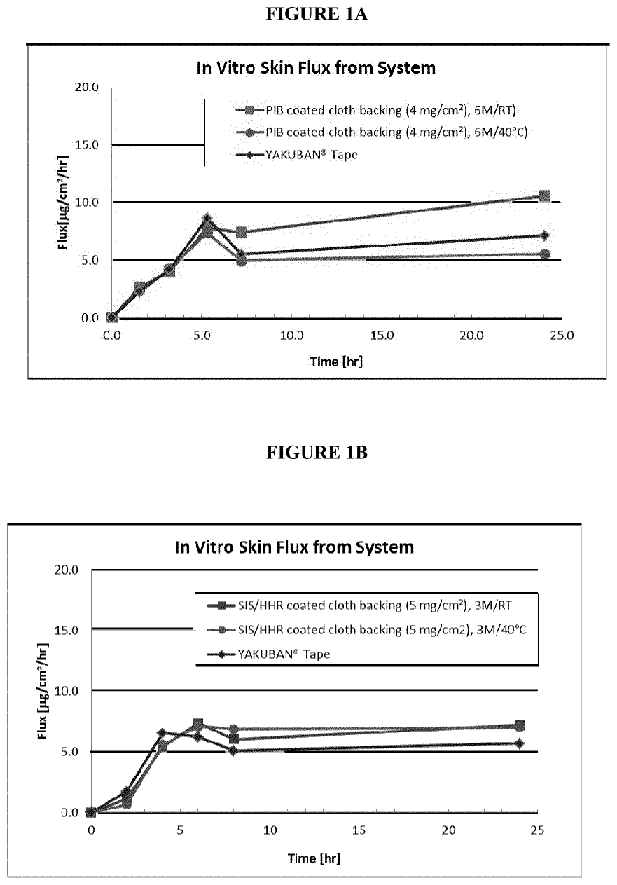

Drug flux from systems stored at room temperature or under accelerated conditions (40° C.) was assessed. Results are shown in FIGS. 1A and B. As seen in the Figures, drug flux from a system with a PIB-coated cloth backing decreased after storage for 6 months at 40° C. relative to drug flux from a system stored for 6 months at room temperature. (FIG. 1A: ♦—YAKUBAN® Tape; X—PIB coated cloth backing (4 mg/cm²) (RT, 6M); ●—PIB coated cloth backing (4 mg/cm²) (40° C., 6M)). On the other hand, drug flux from a system with an SIS/HHR-coated cloth backing maintained relatively constant after storage for 3 months at 40° C. relative to drug flux from a system stored for 3 months at room temperature. (FIG. 1B: ♦—YAKUBAN® Tape; ▪—40% SIS/60% HHR coated cloth backing (5 mg/cm²) (RT, 3M); ●—40% SIS/60% HHR coated cloth backing (5 mg/cm²) (40° C., 3M)). These results confirm that backing layers as described herein maintain their drug flux properties after storage under accelerated conditions.

1. A stretchable, occlusive backing layer for a transdermal drug delivery system for application to skin, comprising a stretchable backing material coated with an occlusive polymer coating comprising a styrene-isoprene-styrene block copolymer (“SIS”) and a C5 to C9 hydrogenated hydrocarbon resin (“HHR”) tackifier, wherein the occlusive polymer coating does not contain drug.

2. The backing layer of claim 1, wherein the stretchable backing material is a stretchable cloth material.

3. (canceled)

4. The backing layer of claim 1, wherein the occlusive polymer coating comprises from 10 to 70% by weight HHR, based on the dry weight of the occlusive polymer coating.

5. The backing layer of claim 1, wherein the occlusive polymer coating comprises from 10 to 90% by weight SIS, based on the dry weight of the occlusive polymer coating.

6. The backing layer of claim 1, wherein the ratio of SIS to HHR in the occlusive polymer coating is from about 10:90 to about 90:10.

7. The backing layer of claim 1, wherein the ratio of SIS to HHR in the occlusive polymer coating is from about 20:80 to about 80:20.

8. The backing layer of claim 1, wherein the occlusive polymer coating further comprises a polyisobutylene polymer.

9. The backing layer of claim 8, wherein the polyisobutylene polymer is present in an amount of up to 25% by weight of the occlusive polymer coating.

10. The backing layer of claim 1, wherein the occlusive polymer coating is applied to the stretchable backing material at a coat weight of from about 1 mg/cm² to about 15 mg/cm².

11. The backing layer of claim 1, wherein the occlusive polymer coating is applied to the stretchable backing material at a coat weight of from about 3.5 mg/cm² to about 11 mg/cm².

12. The backing layer of claim 1, wherein the backing layer has a moisture vapor transmission rate of less than about 60 g/m²/day after stretching to 66% elongation.

13. The backing layer of claim 1, wherein the backing layer has a moisture vapor transmission rate of less than about 100 g/m²/day after storage for 6 months at 40° C.

14. A transdermal drug delivery system for application to skin in the form of a flexible, finite system comprising a stretchable, occlusive backing layer according to claim 1 and a drug-containing polymer matrix.

15. The transdermal drug delivery system of claim 14, wherein the drug-containing polymer matrix comprises a non-steroidal anti-inflammatory drug (NSAID).

16. The transdermal drug delivery system of claim 15, wherein the NSAID comprises flurbiprofen.

17. A method for preparing a stretchable, occlusive backing layer for a transdermal drug delivery system for application to skin that exhibits occlusivity after stretching to an elongation of 20% or after storage for 6 months at 40° C., comprising providing a stretchable backing material with an occlusive polymer coating comprising a styrene-isoprene-styrene block copolymer (“SIS”) and a C5 to C9 hydrogenated hydrocarbon resin (“HHR”) tackifier, wherein the occlusive polymer coating does not contain drug.

18. A method for the transdermal delivery of a drug, comprising topically applying a transdermal drug delivery system according to claim 14 to the skin of a subject in need thereof.

19. The method of claim 18, wherein the transdermal drug delivery system is topically applied to the skin at a joint of a subject in need thereof.

20. The method of claim 18, wherein the transdermal drug delivery system comprises a drug-containing polymer matrix comprising an NSAID.
